(19) United States
(12) Patent Application Publication
(10) Pub. No.: US 2025/0195292 A1
(43) Pub. Date: Jun. 19, 2025

(54) Diaper with cleaning wipe pockets

(71) Applicant: Jeremy Walker, Menifee, CA (US)

(72) Inventor: Jeremy Walker, Menifee, CA (US)

(21) Appl. No.: 18/831,359

(22) Filed: Dec. 11, 2024

Related U.S. Application Data
(60) Provisional application No. 63/608,498, filed on Dec. 11, 2023.

(51) Int. Cl. A61F 13/49

(52) U.S. Cl. CPC A61F 13/49058; A61F 2013/49098

(57) ABSTRACT

A disposable diaper includes an anterior section along a center line axis; a posterior section along the center line axis; an intermediate section between the anterior section and the posterior section along the center line axis; a pocket on the anterior section, an opening of the pocket arranged away from the center line axis; and a cleaning wipe within the pocket.

### CROSS REFERENCE TO RELATED APPLICATION[S]

The present disclosure claims priority to United States Provisional Patent Disclosure Ser. No. 63/608,498 filed Dec. 11, 2023.

### BACKGROUND

The present disclosure relates to a disposable diaper, and particularly to a disposable diaper with a pocket for a cleaning wipe.

Oftentimes, parents may find themselves lacking in supplies during diaper changes.

### SUMMARY

A disposable diaper according to one disclosed non-limiting embodiment of the present disclosure includes an anterior section along a center line axis; a posterior section along the center line axis; an intermediate section between the anterior section and the posterior section along the center line axis; a pocket on the anterior section, an opening of the pocket arranged away from the center line axis; and a cleaning wipe within the pocket.

A further embodiment of any of the foregoing embodiments of the present disclosure includes that the opening is resealable.

A further embodiment of any of the foregoing embodiments of the present disclosure includes that the pocket is secured to a liquid impermeable back sheet layer of the posterior section.

A further embodiment of any of the foregoing embodiments of the present disclosure includes that the pocket is secured to a liquid impermeable back sheet layer of the posterior section.

A further embodiment of any of the foregoing embodiments of the present disclosure includes that the pocket is adjacent to an elastic connector that extends from the posterior section.

A further embodiment of any of the foregoing embodiments of the present disclosure includes that the opening is arranged away from the center line axis and toward the elastic connector.

A disposable diaper according to one disclosed non-limiting embodiment of the present disclosure includes an anterior section along a center line axis; a posterior section along the center line axis; an intermediate section between the anterior section and the posterior section along the center line axis; a first pocket on the anterior section, an opening of the first pocket arranged away from the center line axis; a first cleaning wipe within the first pocket; a second pocket on the anterior section, an opening of the second pocket arranged away from the center line axis; and a second cleaning wipe within the second pocket.

A further embodiment of any of the foregoing embodiments of the present disclosure includes that the first and second pocket is each adjacent to a respective elastic connector that extends from the posterior section.

A further embodiment of any of the foregoing embodiments of the present disclosure includes that the first and second pocket each comprise a wipe pocket pull tab located at the top front corner.

The foregoing features and elements may be combined in various combinations without exclusivity, unless expressly indicated otherwise. These features and elements as well as the operation thereof will become more apparent in light of the following description and the accompanying drawings. It should be appreciated that however the following description and drawings are intended to be exemplary in nature and non-limiting.

### BRIEF DESCRIPTION OF THE DRAWINGS

Various features will become apparent to those skilled in the art from the following detailed description of the disclosed non-limiting embodiment. The drawings that accompany the detailed description can be briefly described as follows:

FIG. 1 is a schematic view of a disposable diaper according to one disclosed non-limiting embodiment.

FIG. 2 is a schematic view of the disposable diaper in a wearable state.

FIG. 3 is a schematic view of a disposable diaper according to another disclosed non-limiting embodiment.

FIG. 4 is a schematic view of the disposable diaper of FIG. 3.

### DETAILED DESCRIPTION

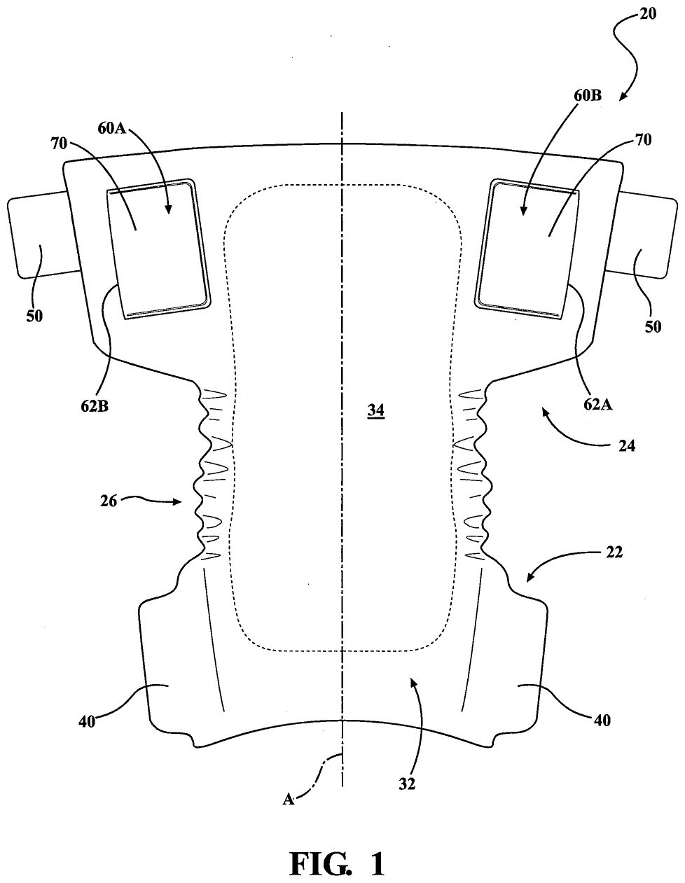

FIG. 1 schematically illustrates a disposable diaper 20 for infants or incontinent persons. The diaper generally includes an anterior section 22, a posterior section 24, and an intermediate section 26 in between. The anterior section 22 is located on the wearer's stomach side when worn and the posterior section 24 is located on the wearer's backside when worn. The disposable diaper 20 may be symmetric with reference to a center line axis A.

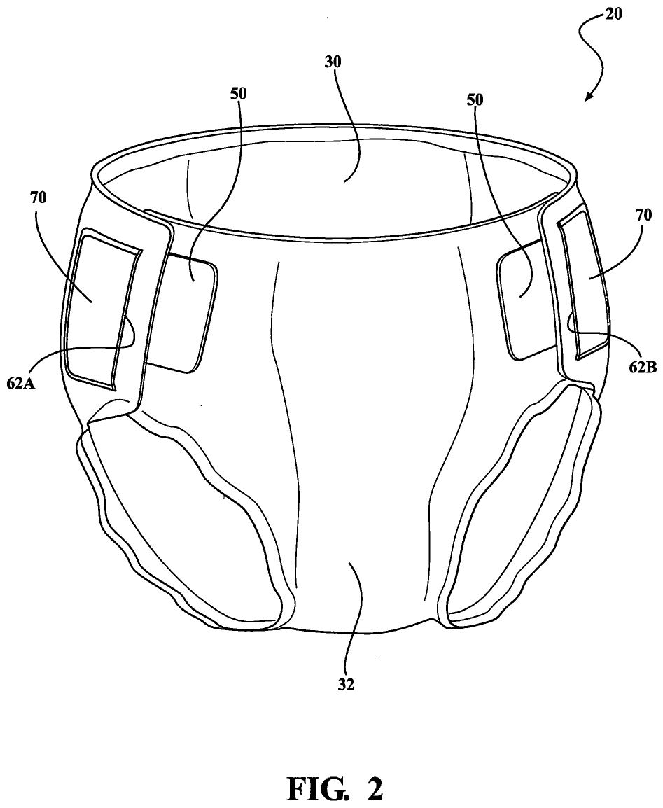

The disposable diaper 20 may include a liquid permeable interior sheet layer 30, a liquid impermeable back sheet layer 32 corresponding to the interior sheet layer 30 (FIG. 2), and an absorbent member 34 fixedly interposed between both the interior sheet layer 30 and back sheet layer 32 to facilitate the absorption of waste material.

The liquid permeable interior sheet layer 30 may be manufactured of woven fabrics, nonwoven fabrics, perforated films and the like. The backs sheet layer 32 may be moisture permeable and liquid impermeable sheet layer able to permeate vapor therethrough and formed of a thermoplastic resin and filler added thereto and stretched and more preferably a sheet layer having a feel as an undergarment. Examples of such liquid impermeable sheet layer are a composite material of film and a woven fabric. The absorbent member 34 may include a fluff pulp as a chief component material and a high molecular water absorbent polymer as a secondary material, or a mixture of a thermoplastic resin, a cellulosic fiber and a high molecular water absorbent polymer subjected to heat treatment. Examples of such high molecular water absorbent-polymer are preferably starch-acrylic (salt) graft copolymer, a saponified material of starch-acrylonitrile copolymer, bridged material of sodium carboxymethylcellulose, acrylic (salt) polymer and the like.

The anterior section 22 may include flaps 40 that extend transverse to the center line axis A to at least partially wrap around the wearer's sides. The posterior section 24 may include elastic connectors 50 that extend transverse to the center line axis A to at least partially wrap around the flaps 40 and secure the disposable diaper 20 to the wearer. The elastic connectors 50 may be a hook and loop fastener, adhesive, etc., that may include an elastic component to at least partially wrap around and thereby secure the posterior section 24 to the anterior section 22.

One or more baby wipe pockets 60A, 60B are secured and/or integrated to the liquid impermeable back sheet layer 32 of the posterior section 24. That is, the baby wipe pockets 60A, 60B are on the outside of the disposable diaper 20 adjacent to the elastic connectors 50 to be arranged adjacent to the wearer's hip joints (FIG. 2).

The baby wipe pockets 60A, 60B each include an opening 62A, 62B that is arranged away from the center line axis A and toward the respective elastic connectors 50. The opening 62A, 62B may be secured via an adhesive, hook and loop, etc., to provide a resealable container for one or more cleaning wipes 70 that are contained within the baby wipe pockets 60A, 60B. The cleaning wipes 70 may be moisturized, and may be manufactured as a mixture of cotton, rayon, polyester, polyethylene, and/or polypropylene and be moisturized with water, a mild detergent containing amphoteric surfactants, etc. Typically, there is one cleaning wipe 70 contained within each baby wipe pockets 60A, 60B, although more than one cleaning wipe 70 may be provided.

The location of the opening 62A, 62B facilitates retrieval of the cleaning wipes 70 while the baby comfortably remains on the soiled diaper while being changed.

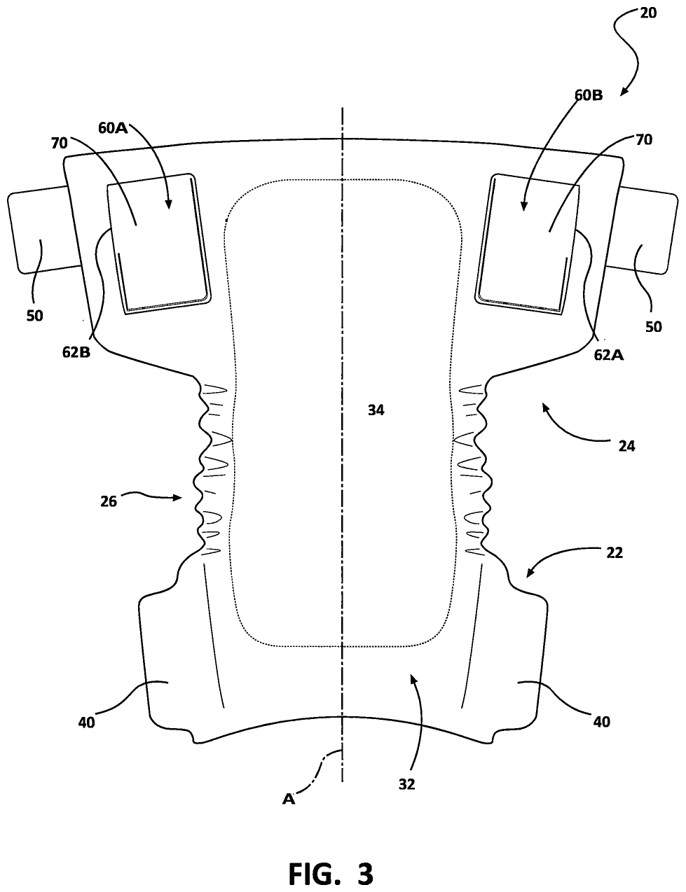

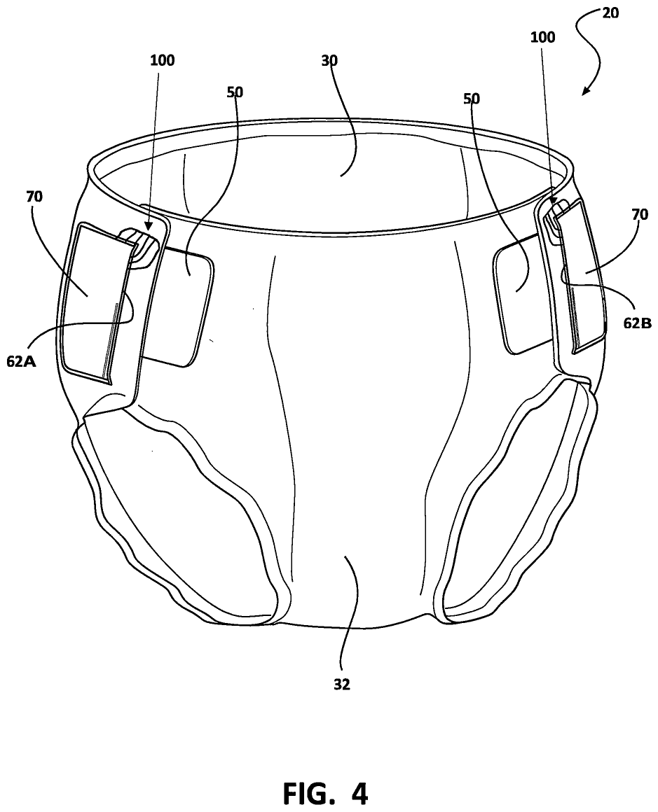

With reference to FIGS. 3 and 4, in another embodiment, each pocket may fit two folded full-size wipes without being too bulky or restricting movement. Each pocket may be, for example, 2¾ inch×3¾ inch in size. The pocket opens from the top front corner so wipes can be accessed in a standing or laying down position. In this embodiment, a wipe pocket pull tab 100 is located at the top front corner.

Although the different non-limiting embodiments have specific illustrated components, the embodiments of this invention are not limited to those particular combinations. It is possible to use some of the components or features from any of the non-limiting embodiments in combination with features or components from any of the other non-limiting embodiments.

The foregoing description is exemplary rather than defined by the limitations within. Various non-limiting embodiments are disclosed herein, however, one of ordinary skill in the art would recognize that various modifications and variations in light of the above teachings will fall within the scope of the appended claims. It is therefore to be appreciated that within the scope of the appended claims, the disclosure may be practiced other than as specifically described. For that reason, the appended claims should be studied to determine true scope and content.

What is claimed is:

1. A disposable diaper, comprising:
an anterior section along a center line axis;
a posterior section along the center line axis;
an intermediate section between the anterior section and the posterior section along the center line axis;
a pocket on the anterior section, an opening of the pocket arranged away from the center line axis; and
a cleaning wipe within the pocket.

2. The disposable diaper as recited in claim 1, wherein the opening is resealable.

3. The disposable diaper as recited in claim 1, wherein the pocket is secured to a liquid impermeable back sheet layer of the posterior section.

4. The disposable diaper as recited in claim 1, wherein the pocket is secured to a liquid impermeable back sheet layer of the posterior section.

5. The disposable diaper as recited in claim 1, wherein the pocket is adjacent to an elastic connector that extends from the posterior section.

6. The disposable diaper as recited in claim 5, wherein the opening is arranged away from the center line axis and toward the elastic connector.

7. A disposable diaper, comprising:
an anterior section along a center line axis;
a posterior section along the center line axis;
an intermediate section between the anterior section and the posterior section along the center line axis;
a first pocket on the anterior section, an opening of the first pocket arranged away from the center line axis;
a first cleaning wipe within the first pocket;
a second pocket on the anterior section, an opening of the second pocket arranged away from the center line axis; and
a second cleaning wipe within the second pocket.

8. The disposable diaper as recited in claim 7, wherein the first and second pocket is each adjacent to a respective elastic connector that extends from the posterior section.

9. The disposable diaper as recited in claim 7, wherein the first and second pocket each comprise a wipe pocket pull tab located at the top front corner.
